(19) United States
(12) Patent Application Publication
(10) Pub. No.: US 2002/0146779 A1
(43) Pub. Date: Oct. 10, 2002

(54) Methods for production of recombinant polypeptides

(76) Inventors: Ian Robert Cottingham, Edinburgh (GB); Colin Martin McKee, Edinburgh (GB); Alan Robert Miller, Edinburgh (GB)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 09/746,945

(22) Filed: Dec. 21, 2000

Related U.S. Application Data
(63) Continuation of application No. PCT/GB99/01907, filed on Jun. 16, 1999.
(60) Provisional application No. 60/098,281, filed on Aug. 28, 1998.

(30) Foreign Application Priority Data
Jun. 26, 1998 (GB) GB 9813912.4

(51) Int. Cl. C12P 21/04

(52) U.S. Cl. 435/69.6

(57) ABSTRACT

Methods for the production of peptides with authentic N-terminal are provided. DNA constructs are also described, which can be used in the production of transgenic animals, which produce the desired peptide in their milk.

The present invention relates to the production of peptides with authentic amino-termini, as well as methods for the production of peptide-acceptor conjugates, by recombinant means.

Peptides consisting of naturally-occurring amino acids can be expressed by recombinant means in a wide variety or organisms. Peptides are defined for the purposes of this application as chains of amino acids joined via peptide bonds and varying in length between three and one hundred amino acids. Currently, peptides are available from three general sources including chemical synthesis, expression from DNA constructs in biological systems and extraction from natural sources. Production of peptides by recombinant means, especially longer peptides over about twenty amino acids, has advantages of cost and ease of purification compared to synthetic chemical routes due to the fidelity of the natural expression process and the avoidance of dangerous chemicals.

One drawback of making peptides by recombinant processes is that due to the general inefficiency of the common expression systems to make peptides, as opposed to larger proteins, fusion protein partners are needed. Fusion partners are used to promote the synthesis of peptides and prevent degradation. These fusion partner proteins can be further exploited on the basis of their specific biological, chemical or physico-chemical properties to aid in purification from the expression system.

Peptides can be made either as amino- or carboxy-terminal extensions of the fusion partner. Generally, peptides made as carboxy-terminal fusions can be cleaved to liberate the natural amino-terminus. Cleavage of an alpha-lactalbumin calcitonin fusion construct with enterokinase is an example of the approach. Here the linker between the carboxy-terminus of the alpha-lactalbumin is joined to the amino-terminus of calcitonin via a short peptide linker which ends in the sequence aspartic acid—aspartic acid—aspartic acid—aspartic acid—lysine. Enterokinase acts specifically on this sequence to proteolytically cleave immediately after the lysine. Therefore, any amino acid can follow the lysine and become the amino-terminus of the liberated peptide. This is only one of a number of different approaches to the specific cleavage of peptide bonds and forms the basis of general methods to cleave fusion proteins to liberate peptides with appropriate amino-termini.

Under some circumstances it is advantageous to express peptides as amino-terminal extensions. A good example of this is where peptides are expressed as amino-terminal extensions to fusion partner proteins capable of generating thioester intermediates. See for example International patent application No. PCT GB98/01281 wherein methods are described for the production of fusion proteins which can be cleaved to produce reactive peptide thioesters, which in turn can then be cleaved by simple chemical reactions to give either free carboxy-termini or amides. However, it is more difficult to obtain native amino-termini on peptides expressed at the amino-terminal end of fusion proteins if expression occurs without secretion and concomitant processing.

Intracellular proteins expressed in recombinant systems, typically E.coli, generally retain the amino-terminal methionine the codon of which is needed to “initiate” tnanslation of messenger RNA into protein. In most cases target peptide sequences will not start with this methionine but rather a different amino acid. Therefore with many target recombinant peptides, especially those destined for human or animal therapeutic uses or specific receptor binding, it is necessary to remove the initiator methionine.

One approach to removing this initiator methionine is to use an expression organism which contains an amino-peptidase enzyme, ie one capable of specifically cleaving only the amino-terminal methionine and not any of the adjacent amino acids, such as that which naturally occurs in the commonly used strains of E.coli. However, in general the known amino-peptidases have limited specificity regarding the penultimate amino acid. For instance, one of the E. coli amino-peptidases which has been extensively characterised only removes the initiator methionine completely if it precedes an amino acid with a short side chain (Hirel, Ph.-Herve et al., Proc. Natl. Acad. Sci. 86:8247-8251 (1989)). Thus peptides starting with any of thirteen of the twenty naturally occurrmng amino acids will retain the initiator methionine. The effect of this specificity is apparent when considering the following examples. Human lutenising hormone releasing hormone starts with a glutanine residue which has a relatively long side chain (when released with a free amino-terminal glutanine will normally cyclise to pyroglutamate). Therefore when LHRH is expressed as an amino terminal extension on an intein in E. coli it is not processed and retains the initiator methionine at its amino terminus. In contrast, human parathyroid hormone (amino acids 6 to 38) starts with an alanine, which has a small side chain, and when expressed in E. coli is processed to give the authentic amino terminus. Thus, the limitations regarding the specificity of the aminopeptidase are not always a problem and, depending on the identity of the penultimate amino-terminal amino acid, it is possible to obtain some peptides as fusion proteins expressed intracellularly in E.coli with the desired amino-terminus. However, it would be an enormous advantage to have a generic method of making peptides with authentic aminotermini regardless of sequence. Such methods form the basis of the present invention and are described below.

There is another issue/problem associated with the production of peptides as the amino-terminus of a fusion protein. Specifically, there is the question of amino-terminal proteolytic degradation. For example, salmon calcitonin expressed in E.coli as the amino-terminal of a fusion protein is proteolytically damaged and direct sequencing of the amino-terminus reveals two inappropriate sequences starting at positions +2 and +6, no authentic amino-terminus and no methionine-extended calcitonin.

The approach described herein is based upon providing an amino-terminal extension of the peptide, which can then be removed either naturally, during the process of secretion, or artificially, eg with a specific cleavage enzyme or chemical reaction, after intracellular expression of the peptide-fusion protein.

Thus, in a first aspect, the present invention provides a method for the production of a peptide with an authentic amino-terminal amino acid, which comprises the step of expressing the peptide as part of a fusion protein, wherein the peptide sequence incorporates a sequence extension at its N-terminus.

In one embodiment, the sequence extension includes an amino acid sequence which can function as a recognition site for a protease. Thus, for example, it is possible to make a small extension of perhaps five to fifteen amino acids, or even a larger peptide or a small protein, at the amino terminus. This will incorporate an amino acid sequence which can function as a recognition site for a protease which will cleave the hybrid peptide at the peptide bond exactly preceding the desired amino-terminal amino acid. Examples of such enzymes are enterokinase, already described above, and Factor X, which cleaves after the recognition sequence isoleucine-glutamate-glycine-arginine. Thus, recovery of the modified peptide, either before or after release from the purified fusion partner complex is carried out by treatment with the appropriate protease to liberate the authentic amino-terminus of the target peptide. Cleavage of the amino-terminal sequence could be based on other proteases with relatively high sequence specificity, such as thrombin or V8 protease but these are likely to cut some target sequences and would need to be tested on a case by case basis.

It is convenient to employ the methods of the present invention in combination with the fusion protein methodology described in PCT GB98/01281. Thus, suitably, at least part of the fusion protein is a molecule capable of catalysing transfer of the peptide, as an acyl moiety, to a suitable acceptor, eg a proximal sulphur atom, to form a thioester. Conveniently this can be achieved by the use of a modified intein sequence, eg one derived from the PI-Sce1 gene from yeast.

Examples of peptides which can suitably be produced using this methodology include Salmon calcitonin, Human calcitonin, Lutenising hormone releasing hormone, Oxytocin, Gastrin neuropeptide Y, Vasopressin, Corticotrophin releasing hormone, Growth hormone releasing hormone, Gastrin, Melanocyte stimulation hormone precurser, Secretin, Thyrotrophin releasing hormone, Amylin, Pramlintide, Substance P, Pancrearic polypeptide, Cholecystokinin, Gastric secretion factor, Savagin, Mastoparin, iaerulein, FMRF aminde, Conotoxins, Brain naturetic peptide, Magainin and related peptides, Galanin and related peptides, Integrelin and related peptides, Glucagon-like peptide 1, Glucagon-like peptide 2, Glucagon related peptides, Calcit anin gene related peptide, Atrial naturetic peptide, Bactolysins, Enhancins and Protectiois.

It is possible to envisage at least two embodiments of this approach. Firstly, the amino-terminal extension a could be cleaved off using the appropriate enzyme whilst the peptide is still attached to the fusion protein partner but after purification on an affinity resin, after selective chromatography or differential precipitation. If, for example, the peptide is pressed as a fusion protein comprising a modified intein (as described in PCT/GE 98101281) having a chitin-binding domain at the carboxy-terminus which acts as an affinity label, the fusion protein can be isolated from lysed E.coli cells and highly purified on a chitin resin matrix. The amino-terminal extension could then be cleaved by incubation of the still-immobilised fusion protein with the appropriate cleavage protease. The released amino-terminal-extension peptide, the protease and buffer salts would then be removed by washing the fusion protein still bound to the resin. Subsequently, the target peptide could be released from the fusion partner by an appropriate treatment such as dithiothreitol. In the majority of cases the thioester intermediate thus generated will then spontaneously hydrolyse in water to give the target peptide, with the desired amino-terminus, without the need for further purification.

The second general approach would be to release the amino-terminal extended peptide from the purified fusion protein, which would be retained by chitin resin, for instance. The cleavage by protease could then be in solution phase, by passing the amino-extended peptide over a column of immobilised protease or by processing in batch using the protease attached to a solid support. The mixture of cleaved amino-extension and target peptide could then be separated by any number of methods based on the relative size, charge, solubility, ligand affinity or other physicochemical difference between the molecules. A simple illustration of this is where a short peptide containing the enterokinase recognition sequence, which would be highly negatively charged compared to a typical target peptide due to the high aspartic acid content, could be resolved from the target peptide by anion-exchange chromatography. In another example, it would be simple to separate an amino-terminal extension peptide of perhaps 10 amino acids from a target peptide of perhaps 30-40 amino acids by reversed-phase chromatography which is a common laboratory method for separating and purifying peptides. A further development would be to include an affinity tag sequence, preferably one comprising a small number of amino acids but possibly a small protein of 100 or more amino acids, which could be captured, before or after cleavage, on an affinity matrix. An example of a short tag is the c-myc sequence which is bound with high selectivity and affinity by a specific (immobilised) monoclonal (Nozaki et al, J.Biochem., 121(3):550-559 (1997)).

A protein such as the chitin binding domain previously mentioned could also be used although a different affinity tag on the intein would be necessary. Finally a sequence rich in amino acids which impart a desirable physicochernical property on the amino-terminal extension, such as many acidic of basic amino acids, six adjacent histidines which create a metal-binding affinity region or even simply a number of hydrophyllic amino acids which in combination with a short (5-15) peptide length will ensure early elution from a reversed-phase column compared to the possibly longer and more hydrophillic target sequence.

An alternative approach is to use chemical cleavage to remove the amino-terminal sequence but again each method needs to be selected after consideration of the target peptide sequence. Cyanogen bromide cleavage after methionine is a possible example of a specific chemical cleavage but this indeed could replace the necessity for using an amino-terminal extension altogether since this method could be used to remove the amino-terminal methionine, the reason requiring the cleavage approach in the first instance. Therefore, if there are no internal methionine sequences in the target peptide cyanogen a peptide could be made with or without an amino-terminal extension. However, even if the sequence does not contain an internal methionine, cyanogen bromide cleavage might be avoided due to the issues surrounding the use and disposal of such a toxic reagent.

Other chemical cleavage methods exist such as a cleavage between adjacent asparagine-glycine residues by treatment with hydroxylamine. However, in general the specificity of proteases and the fact that the target peptide can be of almost any sequence comprising any of the twenty natural amino acids if using enterokinase or Factor X makes this a more universal approach and therefore more attractive hn chemical cleavage methods.

The preparation of recombinant peptides is of growing importance. Industrial scale quantities are often required, eg to make therapeutic agents, antibiotics, targeting delivery-vehicle components for gene therapy, surfactants and many other applications. Material requirements for these uses can range from a few grams per year through kilogram quantities to multi-tonne amounts. In this type of long-term use it is cost-effective to build a dedicated vector, fusion protein, and amino-extension, to choose an expression system most appropriate to the production volume and to develop a dedicated manufacturing process to make a single target peptide. Therefore any variation of the above methods for making peptides with authentic amino-termini could be developed on a case by case basis.

A second use of synthetic peptides is in research where quantities ranging from microgram amounts to a few grams are required. Uses include measuring the effects of substitutions on in vitro or in vivo activity or half-life, as immunogens, as standards for analysis, for epitope mapping or as reagents for other purposes. Normally peptides for such research applications are sourced from specialist suppliers who manufacture them to order by sequential chemical synthesis using so-called solid-phase synthesis. It is also possible for larger facilities to have in-house automated instruments for synthesis although these are relatively expensive with regard to reagents and personnel. Whether peptides are sourced within or without a laboratory or organisation they are expensive and relatively impure because of the inefficiency of chemical synthesis and difficulty in purifying away closely-related contaminants. Factors of cost, purity and the delay in production generally restrict the effectiveness of research using peptides.

On a small scale, certainly up to hundreds of milligrams, the expression of peptides as fusion proteins (for example intein fusion proteins) with amino-terminal extension in E.coli can be adapted to a kit format to allow non-specialists to rapidly and economically make analytical quantities of many different peptides for use in research. Such a kit could contain a number of vectors each based around a promoter, a sequence coding an amino-terminal extension, a poly-linker cloning site, a sequence encoding a thioester promoting fusion protein partner and protein or tag to enable affinity purification. Different but related vectors would contain perhaps two amino-terminal extensions cleaved by different proteases, enterokinase and Factor X for instance. Such kits form another aspect of the invention.

In order to make a given target peptide the first step is to design two complimentary oligonucleotides encoding the peptide sequence and containing restriction sites appropriate for insertion in-frame into the poly-linker site. For peptides over about thirty amino acids it may be necessary to generate two or more sets of complimentary oligonucleotides which can then be ligated together by cloning, PCR amplification or other standard techniques. In the first instance the annealed (double stranded) oligonucleotides might be cloned into the enterokinase vector and expressed in and purified from E.coli. The still-matrix-bound amino-terminal extended peptide-fusion protein would then be incubated in an appropriate buffer with a protein exhibiting enterokinase activity under suitable conditions and for a sufficient time to completely remove the amino-terminal extension. The matrix would then be washed and the buffer changed to promote release of the peptide either in the presence of a thiol alone, to liberate a peptide with a free carboxy group at the carboxy-terminus, or in the presence of thiol and an ammonium salt to liberate an amidated peptide.

Under these conditions the peptide would need no further purification before general use although further purification might be needed, such as reversed-phase HPLC, gel filtration or ion-exchange chromatography, for some applications. It would be advisable to confirm the identity or length of the peptide by an independent method such as mass spectroscopy or direct sequencing. However, this may be redundant as optimised and robust systems are developed. The second vector, encoding the Factor X cleavable extension would be used as a back-up if the target peptide was inappropriately cut by enterokinase which can occasionally cleave at acidic-basic amino acid pairs in some sequences.

Many other configurations of such a peptide-production kit can be envisaged using other methods of cleavage, both enzymatic and chemical or indeed by secretion where natural amino-terminal processing occurs as part of the secretion process. Secretion of peptide fusions is discussed Retail elsewhere in this application. Other manifestations of the peptide kit could include other fusion partners either capable or incapable of producing thioester intermediates and other methods of purification based on either affinity tags or proteins or physicochemical separation. The removal of amino-terminal extensions could also be varied in a number of different ways as described above and including cleavage before or after peptide release using either immobilised or solution phase enzymes.

The ability to make peptides via the well-established and generally sequence independent (some peptides cannot be made by direct chemical synthesis because of their sequence) and inexpensive synthetic oligonucleotide technology also makes other applications requiring families of related peptides more straightforward. For instance, pools of mixed oligonucleotides encoding, for example, peptides with each of a number of different amino acids, or indeed all twenty natural possibilities at a selected position, could be made to allow screening of peptide activities, or other desirable properties. Using the recombinant approach it is now possible to “scan” different positions of a peptide to look for a desired effect using a pool of peptides. Once the assay-property-sensitive position is located then peptides with individual changes can be made and tested to determine the most effective change at a given position. Using the recombinant technology described above such “activity scanning” becomes a few weeks work rather than the long-term, and expensive, programme required by the chemical synthesis of peptides.

A second approach to obtaining authentic amino-termini on peptides expressed at the amino-terminus fusion partners is to engineer secretion of a peptide-fusion protein complex, wherein the fusion partner protein comprises a molecule capable of catalysing transfer of the peptide to a suitable acceptor. For example, an intein can be used to catlyse transfer, as an acyl moiety to a sulphur atom as the acceptor.

Thus, in another aspect, the present invention provides a method for the production Of a peptide with an authentic amino-terminal amino acid, which comprises the step of expressing the peptide as part of a fusion protein, wherein fusion partner protein comprises a molecule capable of catalysing transfer of the peptide to an acceptor, and wherein the peptide incorporates a secretory leader sequence at its amino terminus.

Proteins can be targeted for, secretion in the majority of common expression systems, including bacteria, yeast, mammalian cells in culture, transgenic animals, transgenic plants, insect cells etc, merely by expression of a suitable “secretory leader sequence” at the amino-terminal end of the target protein. This directs the protein towards the secretory pathway and also serves as a target substrate for a specific protease which clips off the leader sequence at a given stage of the secretory process. Thus secreted proteins carry an authentic amnno-terminus which starts immediately after the leader sequence.

Systems designed to direct the secretion of recombinant proteins are well known to those skilled in the art and exist for all commercially developed expression systems (see for example Current Opinion in Biotechnology, 8(5), Editor Edward A. Kohst).

Peptide fusions designed for secretion would be collected from extracellular fluids such as the periplasmic space of bacteria, culture medium from mammalian, plant or insect cells or yeast, tissue such as seeds or leaves from transgenic plants or body fluids such as milk, blood or urine from transgenic animals. The fusion protein is then purified, by specific affinity or differences in physicochemical properties, and cleaved as appropriate, eg by the addition of thiol in the thiolester intermediate based system described in PCT/GB98/01281, or indeed thiol plus ammonia (if amidation is required) to yield authentic target peptide.

Secretion of a peptide fusion protein in the milk of transgenic animals can be used to illustrate the principle of obtaining peptides with authentic amino termini. A DNA construct could be made which comprises a sequence encoding the sheep beta-lactoglobulin promoter, the natural secretory leader sequence for sheep beta-lactoglobulin, the target peptide sequence, the PI-Sce1 intein sequence, the chitin binding domain and the 3′ region again from the sheep beta-lactoglobulin gene. Transgenic animals which stably inherited the transgene would than be made by any of the available methods and mated so that the gene product could be harvested from milk. The peptide intein fusion would then be secreted into milk and the beta-lactoglobulin secretory leader sequence cleaved off to reveal the authentic peptide amino-terminus. Purification of the fusion and cleavage of the peptide at the thioester link would be using methods described in PCT/GB98/01281 (which is hereby incorporated by reference) and elsewhere in this application.

There are many possible variations of fusion proteins which would be capable of carrying a peptide through the expression machinery, being secreted and then forming a thioester intermediate allowing direct peptide release or transfer to an acceptor. These can be designed with a number of different objectives such as improving the efficiency of secretion or blocking of biological activity (to prevent interactions deleterious to the host organism) for instance when expressing anti-bacterial peptides in E. coli (or biologically-active peptides in transgenic animals, or ease of purification.

One consideration with secretion efficiency is the mass ratio of target peptide to fusion partner—if the target peptide is only a few percent of the total mass then much higher overall expression levels are needed to yield substantial quantities of peptide. This issue of size is tout in perspective by considering calcitonin, which is a commercially important target peptide with a mass slightly more than three thousand Daltons, expressed as an amino-terminal extension on the PI-Scel intein—chitin binding domain fusion protein, which has a combined mass of more than sixty-thousand Daltons. Here, only about five percent of the expressed material is target protein. In order to substantially improve this ratio of target peptide to fusion partner it is possible to exploit truncated inteins, so-called mini-inteins, which retain the thioester-transferring-capacity of intein, because they are still capable of self-splicing but have lost the secondary activity normally associated with the unmodified intein. In effect, most inteins are two proteins joined together, a self-splicing domain and a nuclease domain and it has been shown recently that these activities and indeed the two domains, can function separately. An example of this is the intein from Mycobacterium tuberculosis recA which is a 440 amino acid protein which exhibits both splicing and nuclease activities. Genetic engineering has allowed the splicing function to be separated to yield a self-splicing mini-intein of 137 amino acids (Darbyshire, V. et al, Proc. Natl. Acad. Sci, 94:11466-11471 (1997)). The inclusion of a functional mini-intein as a peptide fusion partner will substantially improve the mass ratio benefit of peptide to total fusion protein. In order to exploit mini-inteins for peptide expression it is necessary co disable the carboxy-terminal acceptor site as was done, by a single mutation, for the full sized PI-Sce1 intein. A further improvement in reducing the size of the overall of the overall fusion partner would be to reduce the size of the affinity purification domain and substitute a smaller tag such as a histidine-rich sequence.

The ability, using the present invention, to make recombinant peptides as amino-terminal extensions on proteins capable of generating thioester intermediates, as per the disclosure of PCT/GB98/01281, allows substituent peptide transfer to a variety of different acceptors.

The technology for making peptides as transient thioester intermediates is novel and can be exploited to provide a source of chemically reactive peptides which can then be transferred to different types of acceptors. Peptides with thioesters at the carboxy terminus are desirable reagents because they can be incorporated into a wide range of compounds of both commercial and scientific importance. However, in the past the reactive nature of thioesters has restricted the range of compounds which have been made. In recent years it has been possible to adapt solid-phase chemical synthesis to incorporate thioesters into a target peptide and activated peptides made by this route are of growing importance in peptide synthetic chemistry. However, until now it has not been possible to combine the flexibility, cost-effectiveness and biological fidelity of recombinant peptides with the chemical versatility of thioester chemistry since the multiple reactivities of recombinant peptides with unblocked side groups make it difficult or prohibitively expensive to specifically introduce thioester moieties at the carboxy terminus.

The technology to make recombinant peptides as thioesters is based on the use of protein fusion partners, at the carboxy terminus of the peptide, which are capable of transferring the peptide to the sulphur atom on the side-chain of a proximal cysteine amino acid to form an acyl thioester. The production of such acyl thioester intermediates on peptides, for example generated using a modified self-splicing protein call an intein, is described in PCT/GB98/01281. This application described the generation of recombinant peptides which were either converted to peptides with free carboxy-termini by using a thiol acceptor such as dithiothreitol, to form a transient thioester, subsequently spontaneously hydrolysed by water, or, in the presence of ammonia salts, to form peptides with amidated carboxy-termini. The present application describes an important development of this technology where recombinant peptides can be transferred through thioester intermediates to a whole range of acceptors.

Thus, in an additional aspect, the present invention provides a method for the production of a peptide-acceptor conjugate which comprises:

(i) expressing the peptide as part of a fusion protein;

(ii) release of the peptide from the fusion protein as a thioester intermediate; and

(iii) reaction of the thioester intermediate with an acceptor moiety to form the conjugate.

Alternatively, the reaction can proceed without release of the thioester intermediate, which instead is formed directly with a thiol group on the fusion partner. Thus, in a further aspect the present invention provides provides a method for the production of a peptide-acceptor conjugate which comprises:

(i) expressing the peptide as part of a fusion protein;

(ii) formation of a thioester intermediate directly with a thiol on the fusion partner; and

(iii) reaction of the thioester intermediate with an acceptor moiety to form the conjugate.

In the context of the present invention the term “acceptor” relates to any moiety capable of reacting with the thioester intermediate to form a conjugate, that is to say any moiety comprising a chemical group capable of reactivity towards acyl thioesters. An example of a specific acceptor is the use of other peptides (that is to say, the method will allow for the joining of two peptides). Such peptides could be either naturally occurring peptides or synthetic peptides which are to be joined to the natural peptide produced as part of the fusion protein. The methodology will also allow for modification of the peptide produced by incorporation of particular amino acid derivatives or simple chemical moieties with reactive amines or other suitable chemical functional groups. In addition, it is possible to use an acceptor moiety which incorporates a protected reactive group. The production of the peptide-acceptor conjugate is then followed by removal of the protecting group. The reactive group will then be free to react with the peptide, allowing the generation of cyclic peptides.

The generation of recombinant peptides is exemplified herein in E. coli but the skilled person will appreciate that the methods and ideas described in this application can be utilised and put into practice in any expression system which can be exploited to make recombinant fusion proteins either expressed intracellularly or secreted. The generation of peptides as thioester intermediates is exemplified using a modified self-splicing intein as a carboxy-terminal fusion partner but the same invention applies to any other intein or genetically modified or truncated protein capable of generating such a thioester imtermediate.

In its most basic form, this invention applies to the capture of peptide with carboxy-terminal thioesters which can then be incorporated into any subsequent chemical reaction without interference from other components from the recombinant system. In order to capture the peptide-thioester intermediate, it is necessary to transfer the peptide thioester from the fusion protein, either directly or via a suitable intermediate acceptor, to a thiol intermediate which can be stabilised, or at least placed under conditions which prevent further unwanted reactions such as hydrolysis or self-reactions such as polymerisation. The most direct demonstration of this principle is to purify a peptide-intein fusion protein, using for instance the specific affinity of a component of the fusion partner such a an incorporated chitin-binding domain, and then to incubate under controlled conditions of pH and temperature with a thiol-containing reagent such as dithiothreitol, but not cysteine for reasons described below, such that the peptide is transferred from the cysteine of the fusion protein to the thiol acceptor but not substantially hydrolysed. The peptide-thioester intermediate is then purified under conditions which stabilise the thioester such as reversed-phase HPLC under acidic conditions. The thioester ‘activated’ peptide can then be incorporated into the same types of chemical reactions which are possible with peptide-thioesters made by conventional chemical synthesis.

In addition, it is possible to use other acceptors to capture recombinant peptides directly from the peptide-intein fusion proteins without going via a purified thiol intermediate as described above.

One problem with recombinant peptides is that only the twenty natural amino acids can be incorporated. However, there are many instances where the incorporation of unnatural amino acids such as those with modified side groups or D—chirality are necessary either to mimic natural peptides or to generate molecules with enhanced biological properties or stability. One way in which the recombinant peptide-thioester technology can be used to solve this problem is to join natural peptides via the carboxy-terminus, and a thioester intermediate, to chemically synthesised custom molecules—which can be based on peptide chemistry or simply contain a suitable amine acceptor.

For example, a (short) synthetic peptide can be made with protected side chains and containing modified amino acids or indeed non-amino acid modifications/chemical moieties. Providing that this contains a single unprotected amino group—probably but not necessarily the amino terminus of a peptide—this can be used to “accept” the natural peptide via a thio-ester intermediate to form a stable bond. This principle has been exemplified by using the methyl ester of glycine to “accept” recombinant LHRH (with a retained amino terminal methionine). The methyl ester was used to mimic the properties of an acceptor peptide where the carboxy acid of the “acceptor” is reduced in ionic character as would be observed if it participated in a peptide linkage. Other modifications which might be expected to increase the positive nucleophillic nature of the acceptor amino group would be expected to be equally or more effective in promoting the interaction with the thio ester, to either the intein directly or a thiol intermediate acceptor, and thus facilitate an improved rate and/or efficiency of the reaction with the target recombinant peptide.

A second problem which could be solved by the use of an amine acceptor is the current limitation of the intein system with respect to the terminal amino acid to the target peptide. For reasons intrinsic to the chemistry of the acyl-tnansfer of the peptide to the proximal cysteine of the adjacent intein, cleavage occurs with low efficiency if the target peptide ends in a proline, cysteine or aspargine. In the case of calcitonin, which is a commercially important peptide, the final amino acid is prolinamide and thus synthesis cannot be performed using the intein system. This can be circumvented by synthesis of a truncated calcitonin, missing the final proline, but using prolinamide as an amine acceptor from the intein cysteine. It may be possible to adapt this to target peptides ending in asparagine but those ending in cysteine can be made using cysteine as a direct acceptor as described below.

A second approach for concatenating recombinant peptides using thioester chemistry is to exploit the reaction of thioesters with the sulphur of cysteine to form a thioester intermediate which subsequently rearranges under mild condtions to form a peptide bond. This reaction has been described elsewhere but a major restriction on the general applicability of this approach had been the expense and difficulty of making the peptide-acyl-thioester intermediate. The use of peptide-fusion proteins capable of forming thioester intermediates means that any recombinant peptide can be made as a thioester and then reacted with a wide variety of proteins, peptides or compounds which contain a reduced cysteine with a free amino terminus.

This invention is illustrated by a number of examples. Again a synthetic peptide, with a variety of “unnatural” incorporations, could be made which this time requires a cysteine amino acid at the amino-terminus. This has the advantage that it is not necessary to block any amino groups, within this synthetic peptide but has the disadvantage that this strategy can only work if it is appropriate to introduce a cysteine at the point of joining—if for instance these is a naturally-occurring amino acid or if its presence will not adversely affect the desirable properties of the resultant molecule. However, the “acceptor” molecule can be anything that has a cysteine at the amino terminus. It could be a synthetic peptide, a protein or a non-peptide-based chemical entity. Uses, in addition to joining recombinant and synthetic peptides, include adding labels such as biotin or digitonin to facilitate detection of target peptide interactions in biological systems, adding molecules with adjuvant properties, such as key-hole limpet haemocyanin, carbohydrate structures or lipids, to facilitate antibody production and the attachment of chelating structures to permit radio-labelling of peptides for imaging or radio-therapeutic use. Furthermore, thioester-activated peptides could be linked using the cysteine chemistry to suitably modified chromatography matrices, for example to permit affinity purification of receptors or antibodies, or in the synthesis of peptide complexes, for instance multiple antigen peptide complexes.

Finally, it is also possible that any or all of the acceptors described above to exploit the cysteine chemistry are equally applicable as amine acceptors but the thiol of the cysteine would be substituted by the unmodified amino group.

The invention will now be described with reference to the following examples, which should not be construed as in any way limiting the scope of the invention.

The examples refer to the figures in which:

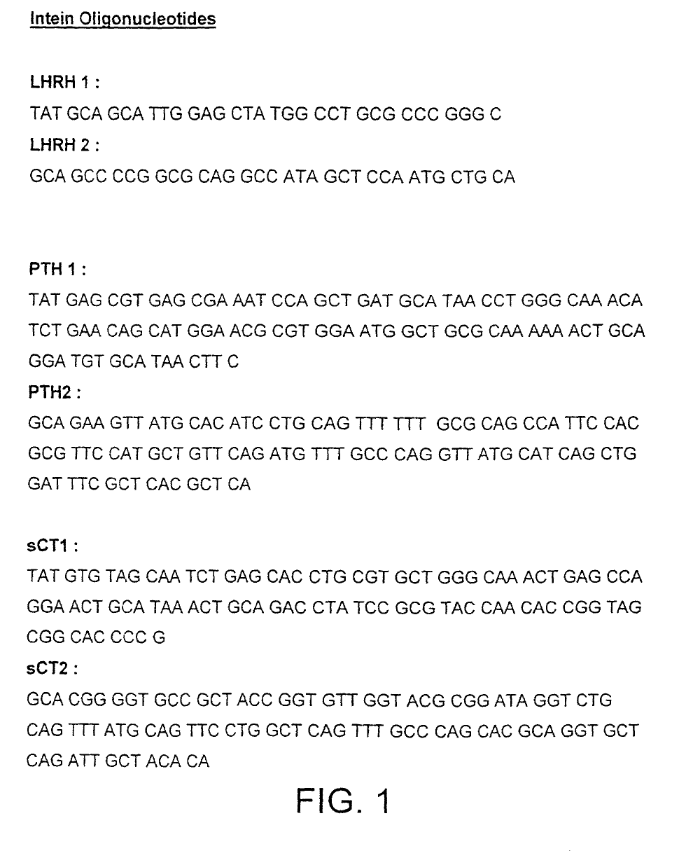

FIG. 1: shows the complementary single stranded oligonucleotides used in the expression of various peptides as described in example 1;

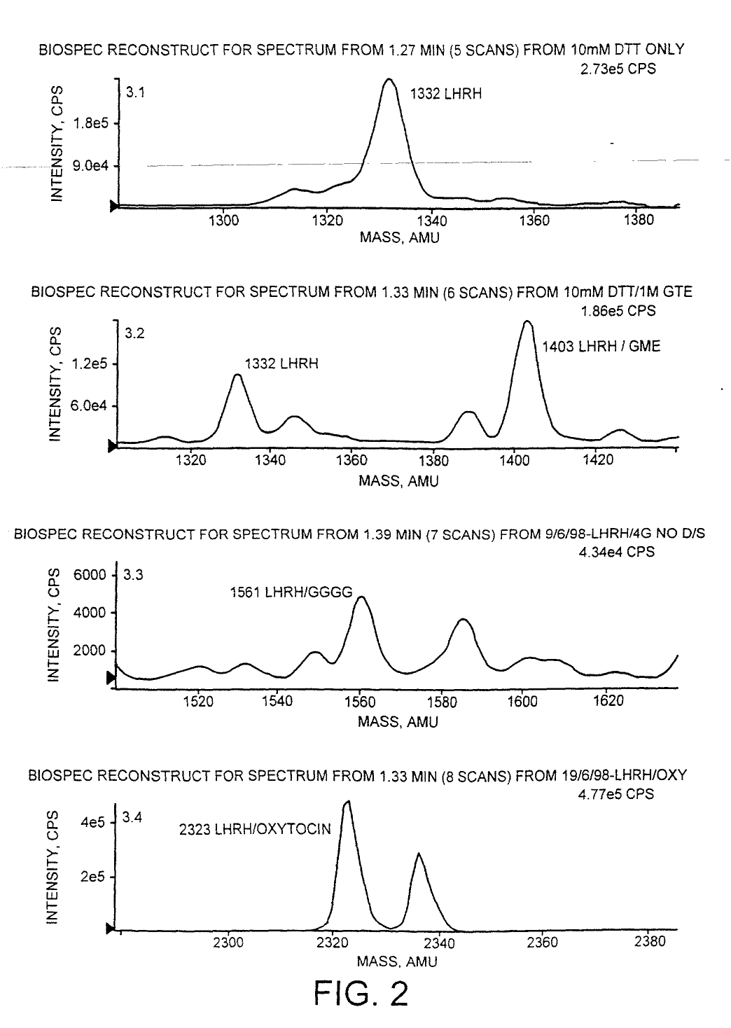

FIG. 2: shows the results of mass spectrum analysis of various recombinant peptides produced according to the methods of the invention;

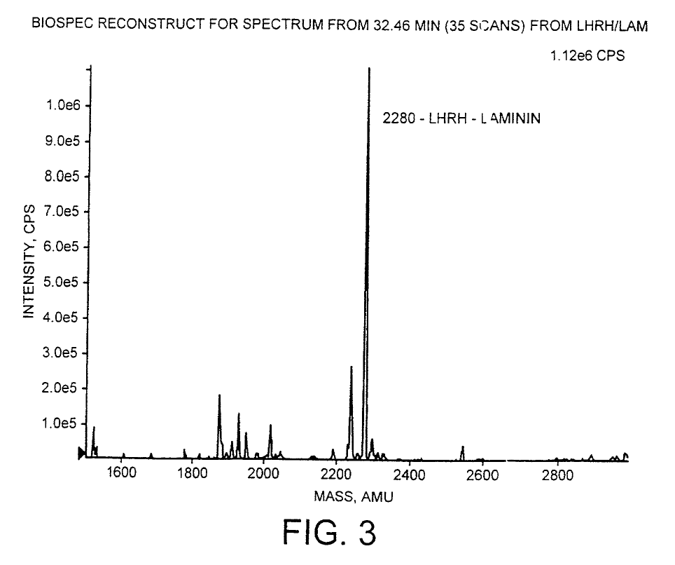

FIG. 3: shows a mass spectrum analysis for a LHRH/LAMIN construct; and

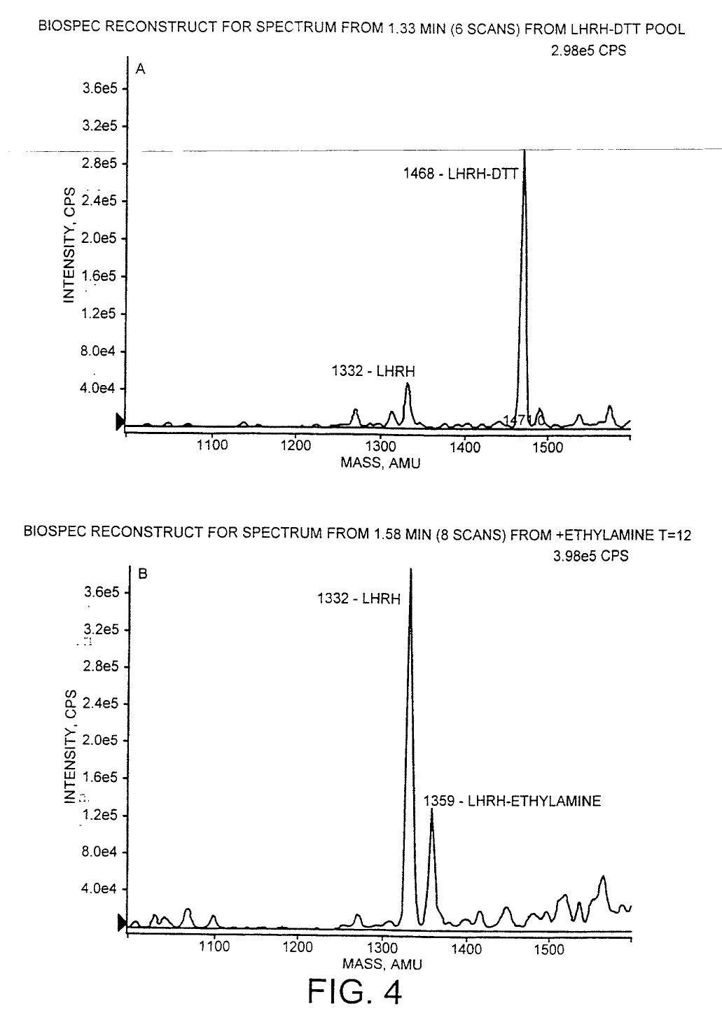

FIG. 4: shows mass spectrum analysis for a peptide-thioester intermediate and the product of subsequent transfer to an acceptor, in this case ethylamine;

### EXAMPLE 1

### Differential Processing of the Amino-Terminus of Peptides Expressed as Amino-Terminal Extensions of an Intein

The three peptides LHRH, Calcitonin and PTH were expressed as amino-terminal extensions of a self-splicing protein, PI-SceI from yeast, which has the carboxy-terminal splicing site disabled by a specific mutation. The intein is also fused at the carboxy-terminal end to a chitin-binding protein which facilitates purification on a chitin chromatography resin. The vector pCYB, which contains the intein construct preceded by a polylinker cloning site is supplied by New England Biolabs. Peptides were expressed from suitable complimentary oligonucleotides cloned into the pCYB vector.

1.1 Expression of LHRH, Calcitonin and PTH

Vector pCYB, containing a Ndel site for translation initiation and a Sapl site directly adjacent to the intein, was used to clone aid express the three peptides. The peptide coding sequences were synthesised as complementary single stranded oligonucleotides (FIG. 1). The codon usage was optimised for expression in E.coli. Annealing of the two strands produced overhangs complementary to the Ndel (5′ end) and the Sapl site (3′ end). The double stranded oligonucleotide was inserted into pCYBl digested with Ndel and Sapl. The expression of the fusion gene is under control of the Ptac promoter and is required by IPTG due to the presence of the laclq gene on the vector.

1.2 Fusion Protein Expression and Purification

The pCYB1 vectors containing the peptides were transfected into DH5a, cells grown under ampicillim selection, induced with IPTG, harvested and lysed by sonication. Expressed fusion proteins were captured on chitin agarose which was washed and then boiled in SDS-PAGE sample buffer The supernatant was run on 16% SDS-PAGE gels. The protein was transferred to PVDF membranes by semidr electrophoretic transfer and visualised with coomassie stain.

1.3 Fusion Protein N-terminal Sequence Analysis

Protein bands were cut from PVDF membranes and sequenced directly by the Edman method. This analysis showed that LHRH was extended by a methionine residue, calcitonin truncated at residues 2 and 6 (Ser2 and Thr6) and that PTH had the authentic N-terminus.

| Peptide | Expected Sequence | Sequence Obtained |
|---|---|---|
| LHRH | pyrHWSYGLR . . . | MQHWSYGLR |
| Calcitonin | CSNLSTCVLGK . . . | SNLSTCV and TCVLGK . . . |
| PTH | SVSGIQLMH | SVSGIQLMH |

### EXAMPLE 2

### Enterokinase Cleavage of a Peptide, Extended at the Amino-Terminus by a Fusion Protein, is Cleaved to Give the Authentic Amino-Terminus

A fusion protein consisting of human alpha lactalbumin, an enterokinase cleavable linker and calcitonin was effectively cleaved with enterokinase. 50 μg of fusion protein in 50 mM Tris/Cl pH 8.0, 1 mM CaCl2 was cleaved with 1 unit of enterokinase activity and the released calcitonin purified by cation exchange chromatography. N-terminal sequence analysis of the purified peptide showed that calcitonin had been specifically cleaved from the fusion protein and that no inappropriate N-terminal proteolysis or modification had occurred. This work is described in further detail in PCT GB98/01281.

### EXAMPLE 3

### Transfer of Recombinant Peptides to Selected Amide and Thiol Acceptors

Methionine-extended LHRH (see Example 1) expressed in the pCYB vector and expressed as an amino-terminal extension of the disabled PI-SceI intein was used to exemplify transfer of peptide-thioester intermediates to a variety of acceptors.

3.1 Transfer to Dithiothreitol and Spontaneous Hydrolysis in Water

Under some conditions transfer to a thio acceptor is followed by hydrolysis to yield the peptide with a free carboxylic acid at the carboxy terminus. In order the demonstrate this the LHRH-Intein fusion was expressed and captured on chitin beads (as described in Example 1) and then incubated in 20 mM Na Hepes pH 8.0, 20 mM DTIT for 16 hrs at 4° C. The column wash was analysed by electrospray mass spectroscopy and the resultant mass spectra was reconstructed to give the mass of the parent ion. The use of DTT to promote cleavage, followed by a prolonged incubation at 4° C., resulted in a parent ion with a mass of 1332Da, which is consistent with Met extended LHRH (FIG. 2.1).

3.2 Transfer to an Amino-Acid Amine Group

In order to demonstrate the general reaction where a recombinant peptide is transferred via a thioester intermediate to an amine group, transfer to the methyl ester of glycine is demonstrated. The methyl ester was used to increase the nucleophillic properties of the amine, and thus counteract the proximal carboxy group. It is expected that a primary amine will give higher reaction efficiency. The HCI salt of glycine methyl ester was dissolved in chloroform:ammonia (90:10 v/v) and mixed for 30 minutes. The resulting NH₄Cl precipitate was removed by centrifugation and the chloroform/ammonia mix dried down to leave the glycine methyl ester with a free amino group. The methyl ester was reconstituted at 1M in 20 mM Na Hepes pH 8.0, 20 mM DTT. LHRi-intein captured on chitin beads was incubated in this solution for 16 hrs at 4° C. The column wash was analysed by ESI-MS. Masses consistent with both methionine-extended LHRH and met-LHRH her extended by glycine methyl ester were observed demonstrating that in addition to hydrolysis of the peptide, transfer to the amino group has occurred (FIG. 2.2).

3.3 Transfer to a Peptide

In addition to using an amine group as an acceptor, it is also possible to use the amino group of a peptide, providing that it does not contain another free amine. Peptide acceptors with reactive side groups would need to be made synthetically and the blocking groups retained until after concatenation. In order to demonstrate the principle of joining peptides, the methionine-extended LHRH was transferred to a tetramer of glcyine, Gly₄.

Gly₄ was reconstituted at 1M in 20 mM Na HEPES pH 8.0, 10 mM DTT and incubated with methionine-extended LHRH-mtein captured on chitin beads for 16 hrs at 4° C. The column wash was then analysed by ESI-MS. Masses consistent with met-extended LHRH, Gly4 and met-LHRH flrter extended by Gly₄ were observed (FIG. 2.3). This confirms that the amino-terminal amine group of Gly₄, which is the only appropriate reactive group of this tetrapeptide, can function as an acceptor for met-LHRH.

3.4 Transfer to a Peptide with a Reduced Cysteine at the Amino-Terminal Position

The methionine-extended LHRH peptide thioester intermediate was reacted with oxytocin presumably at one of the thiols of the two cysteines—oxytocin contains no other reactive groups expected to react with a thioester, for example lysine. It has been reported that an amino-terminal cysteine thiol is capable of reacting with a (peptidyl) acyl thioester and then rearranging to from a peptide bond.

Oxytocin

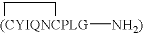

was reconstituted at 1 mM in 20 mM Na Hepes pH 8.0, 10 mM DTT. Methionine-extended LHRH-intein captured on chitin beads was incubated in this solution for 16 hrs at 4° C. The column wash was analysed by ESI-MS. Masses consistent with met-extended LHRH, oxytocin and met-LHRH further extended by oxytocin were observed (FIG. 2.4).

3.5 Transfer of Methionine-Extended LHRH to a Peptide with a Single Cysteine at the Amino-Terminus

A Laminin fragment with a single cysteine was used as an acceptor.

Laminin fragment (CDPGYIGSR-NH₂) was reconstituted at 1 mM in 20 mM Na Hepes pH 8.0, 10 mM DTT. LHRH-intein captured on chitin beads was incubated in this solution for 16 hrs at 4° C. The column wash was analysed by ESI-MS. Masses consistent with met extended LHRH, laminin and met-LHRH further extended by laminin were observed confilring that the MLHRH peptide had been transferred to the laminin fragment. This product was further characterised, after purification by reversed-phase chromatography under standard conditions (C-18 resin with TFA containig buffer and an acetonitrile elution gradient) (FIG. 3).

3.6 Isolation and Reaction of Thioester Intermediate

LHRH-Intein flision was expressed and captured on chitin beads (as described in Example 1) and then incubated in 20 mM Na Hepes pH 8.0, 20 mM DTT at room temperature. The column wash was analysed by electrospray mass spectroscopy, at 30 minute intervals and the resultant mass spectra was reconstructed to give the mass of the parent ion. After 120 minutes the thioester intermediate had accumulated but no significant hydrolysis had occurred (FIG. 4a). This product was purified and reacted with ethylamine at pH 8.0 (as described above). A mixtare of LHRH and LHRH further extended by ethylarnine were observed (FIG. 4). The ratio of hydrolysis:anmine conjugation is a function of both pH and amine concentration and reaction conditions should be optimised accordingly. This shows that the thioester intermediate can be isolated as a stable intermediate and then reacted with a suitable acceptor in a subsequent controlled reaction.

1. A method for the production of a peptide with an authentic amino-terminal amino acid, which comprises the step of expressing the peptide as part of a fusion protein, wherein the peptide sequence incorporates a sequence extension at its N-terminus.

2. A method as claimed in claim 1 wherein the sequence extension includes an amino acid sequence which can function as a recognition site for a protease.

3. A method as claimed in claim 2 wherein the protease is enterokinase, Factor X, thrombin or V8 protease.

4. A method as claimed in any one of claims 1 to 3 wherein at least part of the fusion protein is a molecule capable of catalysing transfer of the peptide to an acceptor.

5. A method as claimed in claim 4 wherein the peptide is transferred as an acyl moiety, to a suitable acceptor, such as a proximal sulphur atom, to form a thio-ester.

6. A method as claimed in claim 4 or claim 5 wherein the fusion protein comprises at least part of a modified intein sequence.

7. A method as claimed in claim 6 wherein the modification of the intein sequence, or part thereof, results in disablement of the self-splicing function.

8. A method as claimed in claim 6 or claim 7 wherein the intein sequence, or part thereof, is derived from the PI-Scel gene from yeast.

9. A method as claimed in claim 6 wherein the fusion protein comprises at least part of a mini-intein.

10. A method as claimed in claim 9 wherein the mnini-intein is the self-splicing mini-intein derived from the Mycobacterium tuberculosis recA intein.

11. A method as claimed in any one of claims 1 to 10 wherein the fusion protein further comprises a label, which allows for identification and/or purification of the fusion protein by affinity, or other chromatographic methods.

12. A method as claimed in any one of claims 1 to 11 wherein the fusion protein further comprises a label, which allows for identification and/or purification of the peptide or non-peptide sequence.

13. A method as claimed in claim 11 or claim 12 wherein the label is an affinity label.

14. A method as claimed in claim 13 wherein the affinity label comprises a specific chitin-binding domain, or part thereof, a repeat of acidic or basic amino acids, a poly-histidine sequence, glutathione synthetase or lysozyme.

15. A method for the production of a peptide-acceptor conjugate which comprises:
(i) expressing the peptide as part of a fusion protein;
(ii) release of the peptide from the fusion protein as a thioester intermediate; and
(iii) reaction of the thioester intermediate with an acceptor moiety to form the conjugate.

16. A method for the production of a peptide-acceptor conjugate which comprises:
(i) expressing the peptide as part of a fusion protein;
(ii) formation of a thioester intermediate directly with a thiol on the fusion partner; and
(iii) reaction of the thioester intermediate with an acceptor moiety to form the conjugate.

17. A method as claimed in claim 15 or claim 16 wherein the acceptor moiety comprises at least one chemical group capable of reactivity towards acyl thioesters.

18. A method as claimed in claim 17 wherein the acceptor moiety is a peptide.

19. A method as claimed in claim 17 wherein the acceptor moiety is an amino acid, an amino-acid derivative or a primary or secondary amine.

20. A method as claimed in claim 19 wherein the amino-acid derivative is proline amide.

21. A method as claimed in any one of claims 1 to 20 wherein the fusion protein is expressed in bacteria, yeast, plant tissue, including whole plants, insect cells, mammalian cells or in a body fluid of a transgenic mammal.

22. A method as claimed in claim 21 wherein the fusion protein is expressed in E.coli or B.subtilis.

23. A method as claimed in claim 22 wherein the fusion protein is expressed in E.coli.

24. A method as claimed in claim 21 wherein the fusion protein is expressed in S.cerevisiae or P.pastoralis.

25. A method as claimed in claim 21 wherein the fusion protein is expressed in chinese hamster ovary cells or baby hamster kidney cells.

26. A method as claimed a claim 21 wherein the fusion protein is expressed in transgenic potato tissue or transgenic corn tissue.

27. A method as claimed in clam 21 wherein the fusion protein is expressed in the milk of a transgenic pig, cow, sheep, goat or rabbit.

28. A method as claimed in claim 21 wherein the fusion protein is expressed in insect cells, e.g. in the S. fugiperda cells.

29. A method as claimed in any one of claims 1 to 28 wherein the sequence coding for the fusion protein also includes a secretory leader sequence.

30. A method as claimed in claim 29 wherein the secretory leader is removed by natural processing enzymes securing secretion.

31. A method for the production of a peptide with an authentic amino-terminal amino acid, which comprises the step of expressing the peptide as part of a fusion protein, wherein fusion partner protein comprises a molecule capable of catalysing transfer of the peptide to an acceptor, and wherein the peptide incorporates a secretory leader sequence at its amino terminus.

32. A method as claimed in claim 31 modified by any one or more of the features of claims 4 to 14.

33. A method as claimed in any one of claims 1 to 32 wherein the peptide is Salmon calcitonin, Hrnman calcitonin, Lutenising hormone releasing hormone, Oxytocin, Gastrin neuropeptide Y, Vasopressin, Corticotrophin releasing hormone, Growth hormone releasing hormone, Gastrin, Melanocyte stimulation hormone precurser,-Secretin, Thyrotrophin releasing hormone, Amylin, Pramlintide, Substance P, Pancreatic polypeptide, Cholecystokinin, Gastric secretion factor, Savagin, Mastoparin, Caerulein, FMRF aminde, a Conotoxin, Brain naturetic peptide, Magainin or a related peptide, Galanin or a related peptide, Integrelin or a related peptide, Glucagon-like peptide 1, Glucagon-like peptide 2, a Glucagon related peptide, Calcitonin gene related peptide, Atrial naturetic peptide, a Bactolysin, an Enhancin or a Protectin.

34. A DNA construct coding for a fusion protein as defined in any one of claims 1 to 20, or 29 to 33.

35. A DNA construct as claimed in claim 34 which is in the form of a vector.

36. A host cell transformed or transfected with a DNA construct as defined in claim 34 or claim 35.

37. A host cell as claimed in claim 36 modified by any one or more of the features of claim 21 to 26.

38. A transgenic, non-human, mammal, which has incorporated in its genome a DNA constct as definedin claim 34.

39. A transgenic manmmal as claimed in claim 38 which is a transgenic pig, cow, sheep, goat or rabbit.

40. A kit comprising one or more reagents for use in the production of a fusion protein as defined in any one or more of claims 1 to 20 or 29 to 33.

41. A peptide-acceptor conjugate as defined in any one of claims 15 to 20


SEQUENCE LISTING (14 sequences)

SEQ ID NO: 1 - protein, 10 aa, Artificial Sequence
  Description of Artificial Sequence  Fusion protein N-terminal
PYRHWSYGLR

SEQ ID NO: 2 - protein, 9 aa, Artificial Sequence
  Description of Artificial Sequence  Fusion Protein N-terminal
MQHWSYGLR

SEQ ID NO: 3 - protein, 11 aa, Artificial Sequence
  Description of Artificial Sequence  Fusion protein N-terminal
CSNLSTCVLGK

SEQ ID NO: 4 - protein, 7 aa, Artificial Sequence
  Description of Artificial Sequence Fusion protein N-terminal
SNLSTCV

SEQ ID NO: 5 - protein, 6 aa, Artificial Sequence
  Description of Artificial Sequence  Fusion protein N-terminal
TCVLGK

SEQ ID NO: 6 - protein, 9 aa, Artificial Sequence
  Description of Artificial Sequence Fusion protein N-terminal
SVSGIQLMG

SEQ ID NO: 7 - protein, 9 aa, Unknown
  PEPTIDE (1)..(9): Oxytocin
CYIQNCPLG

SEQ ID NO: 8 - protein, 9 aa, Unknown
  PEPTIDE (1)..(9): Laminin fragment
CDPGYIGSR

SEQ ID NO: 9 - DNA, 34 bp, Artificial Sequence
  Description of Artificial Sequence  Intein Oligonucleotide (LHRH1)
tatgcagcattggagctatggcctgcgcccgggc

SEQ ID NO: 10 - DNA, 35 bp, Artificial Sequence
  Description of Artificial Sequence Intein Oligonucleotide (LHRH2)
gcagccccggcgcaggccatagctccaatgctgca

SEQ ID NO: 11 - DNA, 106 bp, Artificial Sequence
  Description of Artificial Sequence Intein Oligonucleotide (PTH1)
tatgagcgtgagcgaaatccagctgatgcataacctgggcaaacatctgaacagcatggaacgcgtggaatggctgcgcaaaaaactgcaggatgtgcataacttc

SEQ ID NO: 12 - DNA, 107 bp, Artificial Sequence
  Description of Artificial Sequence Intein Oligonucleotide (PTH2)
gcagaagttatgcacatcctgcagttttttgcgcagccattccacgcgttccatgctgttcagatgtttgcccaggttatgcatcagctggatttcgctcacgctca

SEQ ID NO: 13 - DNA, 100 bp, Artificial Sequence
  Description of Artificial Sequence Intein Oligonucleotide (sCT1)
tatgtgtagcaatctgagcacctgcgtgctgggcaaactgagccaggaactgcataaactgcagacctatccgcgtaccaacaccggtagcggcaccccg

SEQ ID NO: 14 - DNA, 101 bp, Artificial Sequence
  Description of Artificial Sequence Intein Oligonucleotide (sCT2)
gcacggggtgccgctaccggtgttggtacgcggataggtctgcagtttatgcagttcctggctcagtttgcccagcacgcaggtgctcagattgctacaca